(19) United States
(12) Patent Application Publication
(10) Pub. No.: US 2004/0214180 A1
(43) Pub. Date: Oct. 28, 2004

(54) Method and kit for determining quantity of protein

(76) Inventors: Hironori Kobayashi, Kobe-shi (JP); Hideki Ishihara, Kobe-shi (JP)

Correspondence Address: BIRCH STEWART KOLASCH & BIRCH, PO BOX 747, FALLS CHURCH, VA, 22040-0747, US

(21) Appl. No.: 10/423,892

(22) Filed: Apr. 28, 2003

(51) Int. Cl. C12Q 1/68; G01N 33/53; G01N 33/543

(52) U.S. Cl. 435/6; 435/7.1

(57) ABSTRACT

A method for determining the quantity of a protein includes the steps of:

(a) preparing a crude protein liquid containing crude proteins by solubilizing tissue or cells in a pretreatment liquid;

(b) contacting the prepared crude protein liquid with a solid phase to allow the crude proteins to bind to the solid phase; and

(c) determining the quantity of a target protein in the crude proteins binding to the solid phase using a substance which binds specifically to the target protein.

### BACKGROUND OF THE INVENTION

1. Field of the Invention

The present invention relates to a method and a kit for determining quantity of protein, more particularly a method for conveniently determining the quantity of a plurality of kinds of proteins contained in a liquid of solubilized tissue or cells at a time and a kit used for the method.

2. Description of Related Art

It is known that, in cancer tissue, the quantity of specific kinds of proteins increases or decreases as compared with their counterparts in normal tissue. In the case where the type of a disease and/or the seriousness of the disease are/is found to correlate with the kinds of increased or decreased proteins, the detection of the proteins or the determination of the quantity of the proteins existing in tissue is considered to be useful for diagnosing the disease and/or judging the grade of malignancy thereof.

It has been conventionally known that a sandwich ELISA method and a western blotting method can be used for quantitative analysis of a trace of a protein contained in a liquid of solubilized tissue.

The sandwich ELISA method requires two kinds of antibodies, a first antibody which binds specifically to a target protein to be assayed and a second antibody which is different from the first antibody but also binds specifically to the target protein.

In further detail, the first antibody is immobilized in wells of a microtiter plate or on a solid phase support such as a porous membrane or fine particles. A sample containing proteins is allowed to react with the immobilized first antibody. Then the first antibody binds to the target protein corresponding thereto. Subsequently, the second antibody, labeled with an enzyme which can be easily assayed, is further reacted to bind to the target protein binding to the first antibody. Thereafter, the enzyme with which the second antibody labeled is allowed to react with a substrate for the enzyme and the quantity of the resulting product is determined. Thereby the quantity of the target protein is determined.

As described above, the sandwich ELISA method needs two separate specific antibodies (first antibody and second antibody) which bind to one target protein to be assayed (antigen) at different binding sites and which do not affect each other. Also since two antigen-antibody reactions need to take place, the method takes a long time.

For determination of a plurality of target proteins, two specific antibodies are required to be developed. Therefore an enormous amount of labor and time is necessary.

On the other hand, the western blotting method detects and identifies a specific target protein by separating a sample containing the target protein into zones by polyacrylamide gel electrophoresis, transferring the separated sample onto a porous membrane such as a nitrocellulose membrane and a PVDF membrane, and carrying out an antigen-antibody reaction on the membrane.

In detail, the target protein transferred to the membrane is allowed to react with a first antibody which binds specifically to the target protein. Further the first antibody binding to the target protein is allowed to react with a second antibody which is covalently binding to an enzyme capable of being easily assayed and also binds specifically to the first antibody. Thus the second antibody combines with the target protein binding to the first protein. Like the ELISA method, the enzyme binding to the second antibody is allowed to react with a substrate for the enzyme, and the obtained product is detected, so that the existence of the target protein is detected. The quantity of the product is determined and thereby the quantity of the target protein is determined.

In the western blotting method, in place of the second antibody which is covalently binding to the enzyme capable of being easily assayed and also binds specifically to the first antibody, may be used a second antibody which is labeled with a radioactive isotope or a fluorescent substance and also binds specifically to the first antibody. In this case, instead of allowing the enzyme binding to the second antibody to undergo the above-mentioned enzymatic reaction, the existence and quantity of the radioactive isotope or the fluorescent substance are determined by autoradiography or by a fluorescence detector, respectively. The existence or quantity of the target protein is detected or calculated from the obtained results.

As described above, the western blotting method requires the steps of subjecting the sample to electrophoresis and transferring, onto the porous membrane, proteins in the sate separated on the gel according to the molecular weight of the proteins. These steps each need time and complicated work.

Further, in the case of using an SDS-polyacrylamide gel containing an anionic surfactant (SDS) for electrophoresis, the molecular weight of proteins obtained as separated on the gel may be different from the true molecular weight because the binding amount of SDS is generally different to some extent according to the kind of proteins. Therefore, the method is less reliable for identifying the target protein among the separated proteins.

Furthermore, there is no guarantee that zones of the separated proteins are transferred onto the porous membrane linearly to the concentration of proteins contained in the zones. Therefore, the transferred zones of proteins cannot be quantitatively relied upon.

Moreover, after the electrophoresis, the protein transferred on the porous membrane needs to be subjected to two antigen-antibody reactions. The method as a whole takes about dozen or more hours and is not suitable for the purpose of simply determining a plurality of target proteins at the same time.

A solid phase enzyme immunoassay, which is a modified western blotting method, has been reported. According to this assay, a purified antigen protein which binds specifically to a target protein is immobilized directly on a porous nitrocellulose membrane (Japanese Unexamined Patent Publication No. HEI 1(1989)-223352).

The antigen protein immobilized on the membrane is allowed to react with a sample containing the target protein to bind the antigen protein to the target protein. Subsequently, an antibody which binds specifically to the target protein and is covalently binding to an enzyme capable of being easily assayed is reacted with the target protein. Thereby the antibody combines to the target protein binding to the antigen protein.

Similarly to the sandwich ELISA method, the principle of this assay is that the target protein binding to the antibody can be detected by reacting the enzyme bound to the antibody beforehand with a substrate for the enzyme and detecting the existence of the resulting product.

According to this assay, the protein which is immobilized directly on the membrane is neither the antibody which reacts specifically with the target protein nor the target protein itself. Thus, the assay requires one protein which corresponds specifically to one target protein and another specific antibody corresponding to the target protein.

As discussed above, a sample containing a protein to be detected needs to be separated by electrophoresis beforehand as in the western blotting method, needs to be reacted with the second specific antibody after the first antibody specific to the target protein is immobilized on solid phase as in the sandwich ELISA method, or needs to be subjected to the second specific antigen reaction after a protein which specifically corresponds to the target protein other than the antibody is immobilized on the porous membrane as described in Japanese Unexamined Patent Publication No. HEI 1(1989)-223352. Thus, the related-art methods require pretreatment or two or more antibodies or proteins specific to the target protein.

Accordingly, there has been a demand for a method for determining, quickly by a decreased number of steps, the quantity of a plurality of target proteins using only one antibody specific to the target proteins, that is, a method for directly detecting the existence of or determining the quantity of target proteins contained in a crude protein sample.

### SUMMARY OF THE INVENTION

The present invention provides a method for determining the quantity of a protein comprising the steps of:

(a) preparing a crude protein liquid containing crude proteins by solubilizing tissue or cells in a pretreatment liquid;

(b) contacting the prepared crude protein liquid with a solid phase to allow the crude proteins to bind to the solid phase; and

(c) determining the quantity of a target protein in the crude proteins binding to the solid phase using a substance which binds specifically to the target protein.

The present invention also provides a method for diagnosing a disease such as a cancer based on a result obtained by the method for determining the quantity of a protein of the present invention.

These and other objects of the present application will become more readily apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 illustrates an arrangement of aliquots of a standard sample and aliquots of a test sample on a plate used in the method of the present invention;

FIG. 2(a) is a vertical section of an apparatus suitable for carrying out the method of the present invention and FIG. 2(b) is a lateral section thereof;

FIG. 3 illustrates an arrangement of aliquots of a standard sample and aliquots of a test sample on a plate used in Example 1 of the present invention;

FIG. 4 illustrates an arrangement of aliquots of a standard sample and aliquots of a test sample on a plate used in Example 2 of the present invention;

FIG. 5 is a graph showing a calibration line for Cdk2 obtained according to the method of the present invention;

FIG. 6 is a graph showing a calibration line for Cdk4 obtained according to the method of the present invention;

FIG. 7 is a graph showing a calibration line for Cycline E obtained according to the method of the present invention;

FIG. 8 is a graph showing a calibration line for P16 obtained according to the method of the present invention;

FIG. 9 is a graph showing a calibration line for P53 obtained according to the method of the present invention;

FIG. 10 is a graph showing a calibration line for P21 obtained according to the method of the present invention;

FIG. 11 is a graph showing a calibration line for P27 obtained according to the method of the present invention;

FIG. 12 is a graph showing a calibration line for c-myc obtained according to the method of the present invention.

### DESCRIPTION OF THE PREFERRED EMBODIMENTS

The method of the present invention can be carried out according to the following steps:

First, a sample is prepared by diluting a crude protein liquid with a diluent. The crude protein liquid is prepared by pulverizing and solubilizing cells or tissue in a pretreatment liquid using a whirling blender or ultrasonic waves. Alternatively, the crude protein liquid can be prepared by putting cells or tissue in a syringe together with the pretreatment liquid, followed by repeated suction and discharge. As the pretreatment liquid, a buffer solution can be used. The pretreatment liquid may also contain a surfactant, a protease inhibitor and the like.

The buffer agents may be conventionally known ones, and examples thereof include Tris buffers, Good's buffers such as MES, Bis-Tris, ADA, PIPES, ACES, MOPSO, BES, MOPS, TES, HEPES, DIPSO, TAPSO, POPSO, HEPPSO, EPPS, Tricine, Bicine and TAPS, disodium hydrogen phosphate, sodium dihydrogen phosphate, potassium dihydrogen phosphate and the like.

The surfactant is used for destroying cell membranes and nuclear envelopes and extracting intracellular substances to prepare solubilized cells. Examples thereof include Nonidet P-40 (produced by Calbiochem), Triton X-100 (produced by Sigma), deoxycholic acid and CHAPS (3-[(3-chloroamidepropyl)dimethylammonio]-1-propane sulfonate) and the like. The concentration of the surfactant is preferably 1 w/v % or lower, more preferably 1.0 to 0.01 w/v %, still more preferably 0.5 to 0.05 w/v %. Because the concentration of the surfactant is so low, membrane proteins are hardly solubilized, and water-soluble non-membrane proteins are mainly solubilized. If target protein to be assayed is a cancer-associated protein, the crude protein liquid has few impurities such as membrane proteins because most cancer-associated proteins are water-soluble non-membrane proteins. For this reason, the crude protein including the target protein can bind effectively to the solid phase.

The protease inhibitor is used for preventing proteins from being lysed by intracellular proteases which may co-exist in the crude protein liquid containing destroyed cell membranes and nuclear membranes. Examples thereof include a mixture of a metalloprotease inhibitor such as EDTA, EGTA, etc., a serine protease inhibitor such as PMSF, trypsin inhibitor, chymotrypsin, etc., and/or a cysteine protease inhibitor such as iodoacetamide, E-64, etc., and a protease inhibitor cocktail commercially available from Sigma which contains such protease inhibitors premixed.

Preferably, after the cells are solubilized, insoluble substances are removed from the crude protein liquid by centrifugation or by filtration using a filter.

Further, before determining the quantity of a protein according to the method of the invention, it is desirable to determine the total amount of proteins in the crude protein liquid according to a method known to those skilled in the art. The total amount of proteins is determined with reference to bovine IgG using a DC protein kit or the like.

Preferably, after the total amount of proteins is determined, the crude protein liquid is diluted 10- to 100-fold and subjected to the steps described in the following paragraphs. The later-mentioned diluents are usable for dilution. If the crude protein liquid is used as a sample after it is diluted, the surfactant, for example, in the crude protein liquid is also diluted and the concentration thereof in the sample is low. For this reason, the surfactant does not inhibit crude proteins in the sample from binding to the solid phase, and thus the crude proteins can bind efficiently to the solid phase.

Next, the sample is brought into contact with the solid phase so that the crude proteins in the sample bind to the solid phase. A porous membrane, beads and the like may be used for the solid phase. As beads, latex particles and magnetic particles are usable. The introduction of a hydrophobic bonding group, an ion exchanger, a substrate, an antibody and the like into the solid phase allows the proteins to bind efficiently thereto. The antibody introduced in the solid phase should not bind specifically to the target protein but should bind to the crude proteins including the target protein. Preferably the hydrophobic bonding group is introduced in the solid phase.

Next, the target protein in the sample binding to the solid phase is allowed to bind to the substance which binds specifically to the target protein. As the substance which binds specifically to the target protein, an antibody and nucleic acid can be used.

For example, in the case where the target protein is selected from the group consisting of actin, Cdk1, Cdk2, Cdk4, Cdk6, Cycline B, Cycline D, Cycline E, P16, P21, P27 and C-myc, antibodies which bind specifically to these proteins can be obtained by a ordinary method by giving part or all of the respective proteins to an animal such as a goat, rabbit, rat, mouse, pig, sheep or chicken.

The nucleic acid which binds specifically to the target protein can be obtained by aptamer technology.

Is now described an example where an antibody is used as the substance which binds specifically to the target protein.

A first antibody which is labeled or has a site reactive to a label and which is specific to the target protein to be assayed is brought into contact (mixed) with the solid phase to which the crude proteins are binding so that the first antibody binds to the target protein.

As a labeled antibody, is usable a labeled antibody known in the field of art. In detail, the labeled antibody means an antibody labeled with a fluorescent substance or with an enzyme for labeling.

The fluorescent substance for labeling may be fluorescein, coumarin, eosin, phenanthroline, pyrene, rhodamine or the like, among which fluorescein is preferred.

The enzyme for labeling may be α-galactosidase, alkaline phosphatase, peroxidase or the like, among which peroxidase is preferred.

The antibody having a site reactive to a label may be an antibody having a site reactive to a label known in the field of art. For example, in a site of an antibody reactive to a label substance FITC (fluorescein isothiocyanate), an isothiocyanate moiety of FITC reacts and binds with an amino group of the antibody, so that the antibody is labeled with fluorescein.

The first antibody is allowed to bind to the target protein by contacting (mixing) the first antibody with the solid phase to which the crude proteins are binding, followed by reaction at room temperature for 15 to 30 minutes.

The first antibody may be used in the form of a solution, preferably in the form of a solution in a Tris-HCl buffer (pH7.4). The solution may further contain sodium chloride, ATP and DTT. The amount of the first antibody in the solution may be adjusted as appropriate so that the first antibody is supplied in an amount larger than the estimated amount of the target protein, taking into consideration the previously measured total amount of proteins in the sample.

Subsequently, the first antibody, unreacted, is removed by washing.

TBS-T (250 mM Tris, 150 mM sodium chloride, 0.05% Tween 20) or the like may be used as a washing liquid. Washing may be performed one or more times, a plurality of times.

If an unlabeled antibody is used as the first antibody, a label is allowed to act at the reactive site of the first antibody to label the first antibody. For example, if a commercially available biotinylated antibody is used as a first antibody, the biotinylated antibody can be detected by reacting the biotinylated antibody with avidin having a detectable label (FITC-labeled avidin, HRP-labeled avidin, rhodamine-labeled avidin, etc.).

Subsequently, the amount of the label binding to the target protein is measured according to a method known in the field which is suitable for the label.

In detail, where the first antibody is labeled with a fluorescent substance, the amount of fluorescence from the fluorescent substance is measured. For example, the fluorescent substance is excited with a specific wavelength and the fluorescence therefrom is detected by a fluorescent image analyzer. The wavelength of excitation light is varied depending upon the type of the fluorescent substance used, but if the fluorescent substance is fluoroscein, it is excited by irradiation with 488 nm wavelength.

If the first antibody is labeled with an enzyme for labeling, the enzyme may be reacted with a substrate which generates an optically detectable product by reaction with the enzyme, and the amount of the generated product may be optically measured.

The substance which can be optically detected through the reaction with the label enzyme means a substance whose existence can be detected by measuring fluorescence, absorbance, scattered light intensity, transmitted light intensity and the like. Examples thereof may be dyes such as ECL-plus and TMB (tetramethylbenzine), luciferin and the like, among which ECL-plus is preferred. For example, if the label enzyme is peroxidase, the substance which can be optically detected through the reaction with the enzyme may be ECL-plus. The substrate for the label enzyme may be selected as appropriate according to the type of the enzyme used.

Subsequently, the amount of the target protein is calculated from the amount of the label with reference to a pre-produced calibration line.

If the antibody is labeled with the fluorescent substance, the measured amount of fluorescence is applied to the pre-produced calibration line of the amount of the protein with respect to the amount of fluorescence which has been obtained beforehand by the same procedure as described above of a known amount of the pure protein. Thereby the amount of the target protein contained in the solubilized sample of tissue or cells which is binding to the solid phase can be calculated.

If the antibody is labeled with the enzyme, the measured amount of the product generated through the reaction with the enzyme is applied to the pre-produced calibration line as mentioned in the previous paragraph. Thereby the amount of the target protein contained in the solubilized sample of tissue or cells which is binding to the solid phase can be calculated.

For determining the quantity of a plurality of proteins, the use of antibodies labeled with the same label specific to the proteins is preferable because the quantity of the proteins can be determined by a fluorescence detector using only one wavelength for exciting the label.

In the case where a porous membrane is used as the solid phase, the crude proteins in the sample are immobilized on the membrane by putting the sample in wells of a plate with the porous membrane disposed at the bottom of the wells and sucking the sample by applying a negative pressure from a porous membrane side of the plate.

The porous membrane may preferably be a hydrophobic porous membrane capable of binding with proteins by hydrophobic bonds. Examples thereof include a PVDF (polyvinylidene fluoride) hydrophobic membrane, a nylon membrane (charged), nitrocellulose and the like.

The porous membrane at the bottom of the wells has pores with a diameter of 0.1 to 10 μm, preferably 0.1 to 0.5 μm. The suction from the porous membrane side of the plate may be performed at about 50 to 1,000 mmHg, preferably about 100 to 300 mmHg, for about 5 to 120 seconds. The size of the wells may be selected in consideration of the sum of the bottom area of the wells and easy suction by the negative pressure from the membrane side.

The proteins are immobilized on the hydrophobic porous membrane by hydrophobic interaction.

Preferably, the hydrophobic porous membrane may be subjected to pretreatment, for example, immersion in a transfer buffer (48 mM Tris, 39 mM glycine, 20% methanol, SDS and 80% water), before use.

The amount of the sample put in the wells in aliquots can be easily selected by those skilled in the art according to the total amount of the proteins in the sample and the kind of the target protein whose quantity to be determined. At this time, the sample is preferably put in each well in such an aliquot that contains the proteins in a smaller amount than can be immobilized on the predetermined area of the porous membrane at the bottom of the well. If the sample is likely to contain the proteins in a larger amount than can be immobilized on the predetermined area of the porous membrane at the bottom of the well, for example, the sample may be diluted with a diluent such as Tris buffer, phosphate buffer or water in order that the total amount of the proteins in the sample put in each well is adjusted to the amount that can be immobilized on the predetermined area of the porous membrane at the bottom of the well or less.

For example, if a sample of solubilized HeLa cells containing 1 μg/mL of proteins in total is used, the bottom area of the well is 24 mm² and the target protein is Cdk1, Cdk2, Cdk4, Cycline B, Cycline D, Cycline E, P16, P21, P27, C-myc and the like, then the aliquot of the sample is about 100 μL per well.

On the other hand, if the target protein is actin, the sample is a sample of solubilized HeLa cells containing 0.5 μg of proteins in total and the bottom area of the well is 24 mm², then the aliquot of the sample is about 100 μL per well. At this time, since the content of actin in the sample of solubilized HeLa cells is larger than that of other target proteins, the sample may be diluted with a diluent such as Tris buffer, phosphate buffer or water in order to adjust the total amount of the proteins.

Preferably the plate has one or more wells. If the plate has a plurality of wells, it is possible to immobilize the same sample on the porous membrane at the bottom of the wells, put a first antibody specific to different proteins and thus determine the quantity of the proteins in the sample. Also it is possible to immobilize different samples on the porous membrane at the bottom of the wells, put a first antibody specific to one protein and determine the quantity of the protein in the different samples.

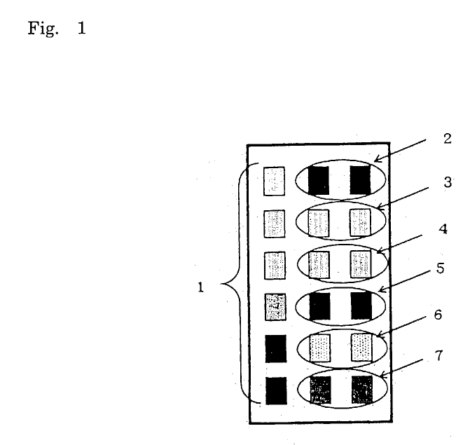

A pure product of the target protein may be put in the wells of the plate in concentration gradient. For example, each row may have six wells in a plate, as shown in FIG. 1. For determining the quantity of the target protein using this plate, the pure product is put in one row of wells in amounts of 0 ng (as a background not containing the protein), 5 ng, 12.5 ng, 25.0 ng, 37.5 ng and 50 ng. This row is referred to as a standard series 1. Such immobilization of the pure product on the same plate is preferable because the pure product is allowed to react under the same conditions as samples 2 to 7 to be determined which are immobilized on the same plate and a highly accurate calibration line can be produced on the basis of the results obtained from the pure product having the concentration gradient (see FIG. 1).

Optionally a blocking liquid is added into the wells for preventing the proteins immobilized on the porous membrane from unspecifically binding to external factors during reaction with the antibody and giving rise to measurement errors. The blocking liquid is preferably added after the immobilization of the proteins in the sample on the porous membrane.

As the blocking liquid, 4% BSA (bovine serum albumin) may be used. Other known blocking liquids may be utilized. After the blocking liquid is added into the wells, the resulting mixtures in the wells are allowed to stand and react at room temperature for 0 to 60 minutes. Thereafter, the blocking liquid is removed by the suction from the porous membrane side of the plate as described above.

Regarding the solid phase to which the proteins are binding;

(1) the first antibody specific to the target protein to may be allowed to bind to the target protein;

(2) the unreacted first antibody may be removed by washing;

(3) a second antibody which is labeled or has a site reactive to a label and which is specific to the first antibody may be allowed to bind to the first antibody;

(4) in the case where the second antibody, unlabeled, is used, a label may be allowed to act on the second antibody for labeling the second antibody.

(5) the amount of the label binding to the target protein may be measured; and

(6) the quantity of the target protein may be calculated using the amount of the label with reference to the pre-produced calibration line.

The second antibody which is labeled or has a site reactive to a label may be labeled in the same manner as the above-described first antibody or may have the same site reactive to a label as the first antibody. So long as the second antibody has specificity to the first antibody, it is unnecessary to use different second antibodies for different target proteins. For determining the quantity of different target proteins, a single second antibody may be used in common. Where a single first antibody is used, a single second antibody specific to the first antibody may be used.

The present invention further provides a method for diagnosing a cancer such as stomach cancer, intestinal cancer, breast cancer, lung cancer, esophagus cancer, prostate cancer, liver cancer, kidney cancer, bladder cancer, skin cancer, uterine cancer, brain tumor, osteosarcoma or myeloma with the results for the quantity of the protein determined according to the present invention. For example, if the quantity of proteins such as actin, Cdk1, Cdk2, Cdk4, Cdk6, Cycline B, Cycline D, Cycline E, P16, P21, P27, C-myc or the like increases or decreases, there is the possibility that a patient has stomach cancer or intestinal cancer.

Specifically, in the case of intestinal cancer, the quantity of P21 decreases and the quantity of Cycline B increases.

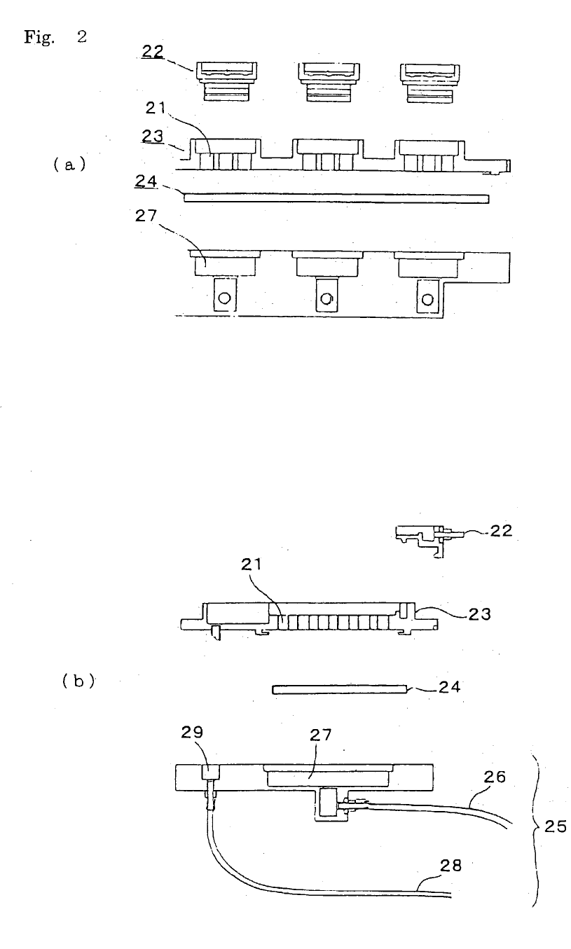

The method of the present invention can suitably be carried out with use of a sample analyzer as shown in FIGS. 2(a) and 2(b) which includes:

a support having a plurality of units each having a plate 23 provided with a plurality of perforated wells 21 and a liquid supply path 22 at an end, and a hydrophobic porous membrane 24 disposed at the bottom of the wells, the plate 23 and the hydrophobic porous membrane 24 being-detachably mounted on the support, and

a suction mechanism 25 for sucking a liquid supplied into the wells from the liquid supply path in a direction to the bottom of the wells.

The sample analyzer can be used for carrying out the method of the present invention in the following manner:

A sample liquid or a specimen liquid is put into the wells of the sample analyzer and sucked in the direction to the bottom of the wells by negative pressure using the suction mechanism, so that the liquid is removed from the wells. Particularly, the liquid in the wells is removed therefrom by the negative pressure by the suction mechanism, for example, a suction groove 27 connected to a solid phase drain 26 and a discharge groove 29 connected to an overflow drain 28. As a result, the proteins in the sample liquid or the specimen liquid are immobilized on the hydrophobic porous membrane.

The wells of the sample analyzer in which the proteins are immobilized to the hydrophobic porous membrane at the bottom are fed with a solution containing the antibody and are allowed to stand under pre-set conditions to progress an antigen-antibody reaction or the like in the wells. After the reaction, the liquid in the wells is removed using the suction mechanism of the sample analyzer as described above.

For washing the inside of the wells, a washing liquid or the like is introduced into the wells by the liquid supply path of the sample analyzer and then removed from the wells using the suction mechanism of the analyzer.

Thus the use of the suction mechanism of the analyzer enables liquid to be removed from the wells of the plate in a short time, and the method of the present invention can be carried out easily and quickly.

A number of components used for the method for determining the quantity of the present invention may be packaged beforehand into a kit. The kit includes at least the pretreatment liquid for solubilizing tissue or cells; the solid phase for binding thereto the crude proteins in the crude protein liquid by solubilizing tissue or cells; and the substance which specifically binds to the target protein in the crude protein liquid. The kit may include as required the diluent for diluting the crude protein liquid and the washing liquid for washing the solid phase. In the kit, the pretreatment liquid, the solid phase and the substance which binds specifically to the target protein are separately packed.

### EXAMPLES

For further detailed explanation of the method of the present invention, examples of protocols are shown below.

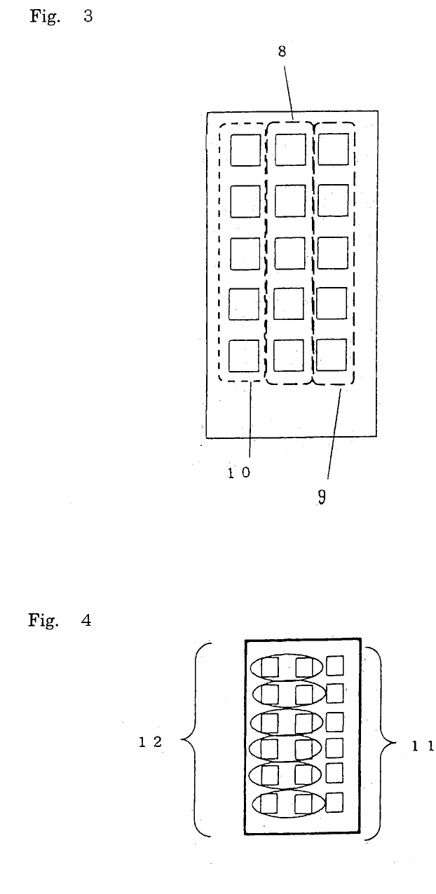

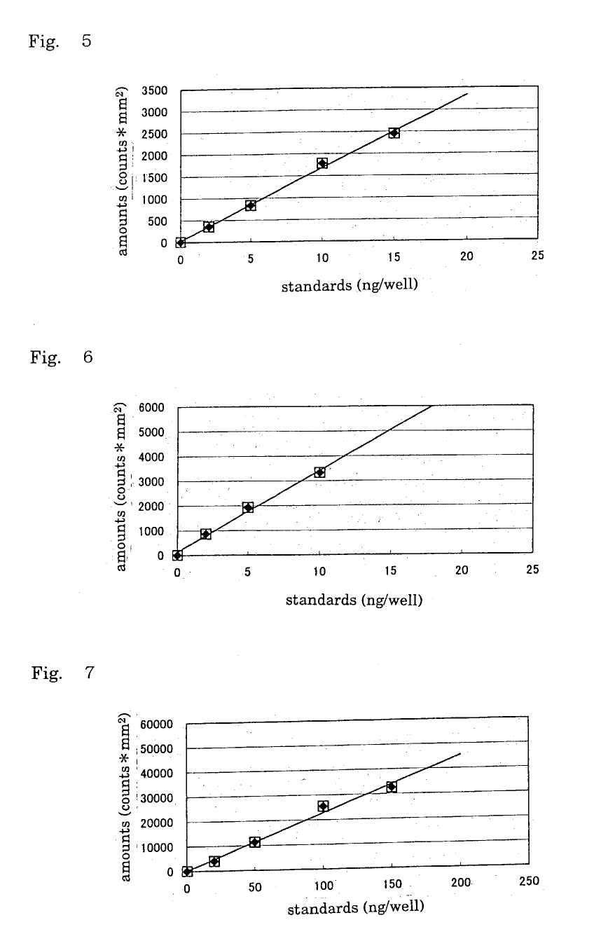

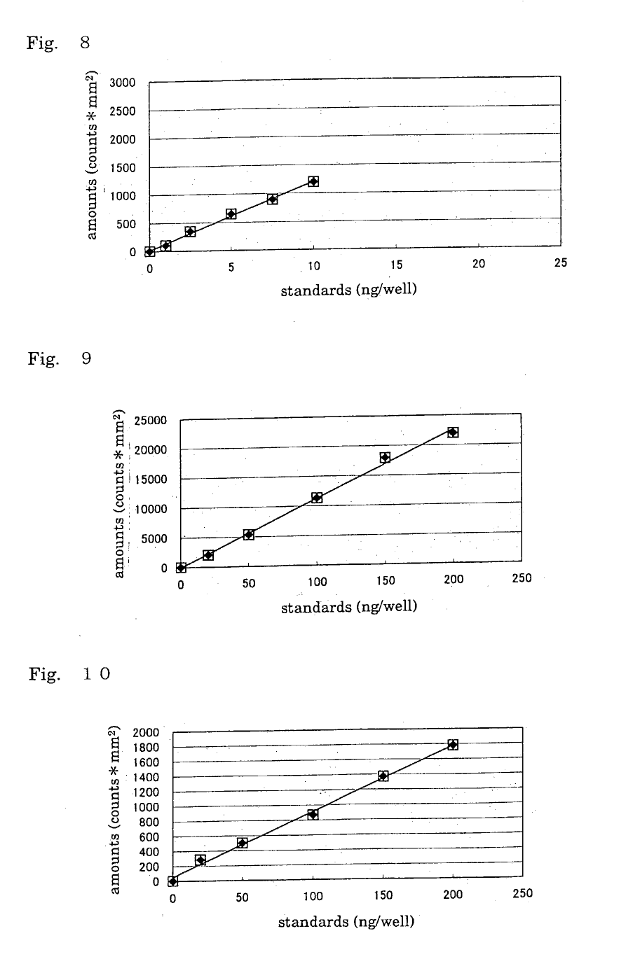

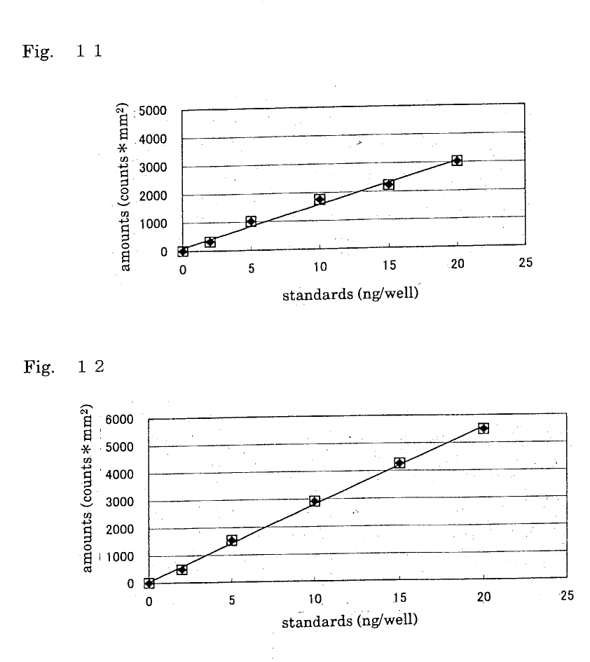

### Example 1 (see FIG. 3)

1. In an ice bath, HeLa cells (carcinoma cells of uterine cervix) were lysed in a lysis buffer containing 0.1 w/v % NP-40 (produced by Calbiochem), 50 mM Tris-HCl, pH 7.4, 5 mM EDTA, 50 mM sodium fluoride, 1 mM sodium orthovanadate and a protease inhibitor cocktail (Sigma), under the condition of 1×10⁷ cells/5 mL the lysis buffer (pretreatment liquid), by 10 times repeated sucking and discharging with a 5-mL syringe provided with a 23G needle. A cell lysate was thus prepared.

2. Insolubles were removed by centrifugation at 4° C. at 15,000 rpm for 5 minutes. The total amount of proteins contained in the supernatant was measured by a DC protein kit (Bio-Rad) using bovine IgG as reference.

3. A sample was prepared by dilution with TBS (50 mM Tris-HCl, 100 mM NaCl; pH7.4) containing 0.001% NP-40 so that the total amount of proteins became 1 μg/100 μL.

4. A PVDF (polyvinylidene fluoride) membrane was immersed in a transfer buffer (48 mM Tris, 39 mM glycine, 20% methanol, 0.1% SDS and 80% water) for pretreatment.

5. A plate provided with 15 wells (5 lines×3 rows) each having a fixed bottom area (24 mm²) was attached on the pretreated PVDF membrane and fixed so that the bottom of the wells was formed of the PVDF membrane.

6. Each well in 5 lines×1 row of the plate was fed with 100 μL of a mixture of the sample of 1 μg/100 μL of the total proteins in TBS containing 0.001% of NP-40 and a rabbit IgG antibody in an unknown concentration in TBS containing 0.001% of NP-40 (Sample Series 8).

7. Each well in another 5 lines×1 row of the same plate was fed with 100 μL of a cultured cell (HeLa cell) sample of 1 μg/100 μL of the total proteins in TBS containing 0.001% of NP-40 (Negative Control Series 9).

8. Each well in another 5 lines×1 row of the same plate was fed with 0 ng/100 μL, 2 ng/100 μL, 4 ng/100 μL, 8 ng/100 μL and 16 ng/100 μL of the rabbit IgG antibody in TBS containing 0.001% of NP-40 and 1 μg/100 μL of BSA, 100 μl each. (Standard Series 10).

9. After all the wells of the plate were fed with the liquids, the wells were sucked from the bottom of the wells, that is, from the back face of the membrane at a negative pressure of about 200 mHg for about 15 seconds.

10. Subsequently, all the wells of the plate were fed with a washing liquid (TBS-T:250 mM Tris, 1.5 aqueous solution of sodium chloride, 1.0% Tween 20), and the wells were sucked from the bottom of the wells at a negative pressure of about 500 mHg for about 30 seconds.

11. All the wells of the plate were fed with a blocking liquid (TBS-T, 4% BSA), 100 μL each, and allowed to stand at room temperature for about 30 minutes. Thereafter, the wells were sucked from the bottom of the wells at a negative pressure of about 500 mHg for about 15 seconds. Then, all the wells of the plate were washed in the same manner as in step 10.

12. All the wells of the plate were fed with an anti-rabbit antibody (1.5 mg/mL TBS-T solution of 1/4000 FITC anti rabbit IgG) labeled with FITC (fluorescent isothiocyanate) which binds specifically to the rabbit IgG antibody, 100 μL each, and were allowed to stand at room temperature for about 30 minutes. Thereafter, the wells were sucked from the bottom of the wells at a negative pressure of about 500 mHg for about 30 seconds. Then, all the wells of the plate were washed in the same manner as in step 10.

13. The PVDF membrane was removed from the plate, washed with distilled water and dried at room temperature for about 15 minutes. Thereafter, the PVDF membrane was analyzed by a fluorescence detector to detect fluorescence emitted from the label substance binding to the protein adsorbed on the membrane in a size corresponding to the bottom area of each well.

14. The quantity of the protein in the sample was calculated from the average fluorescence intensity obtained from the sample series 8 (5 wells), that obtained from the negative control series 9 (5 wells) and the fluorescence intensity obtained from the wells at different concentrations in the standard series 10 (5 wells). The average fluorescence intensity obtained from the negative control series 9 (5 wells) was considered a background fluorescence due to autofluorescence of the membrane because the IgG protein was not adsorbed on the membrane.

The net fluorescence intensity of the sample excluding the background fluorescence is calculated by the following equation:

(Net fluorescence intensity of sample)=(Average fluorescence intensity obtained from the sample series)—(Average fluorescence intensity obtained from the negative control series).

In the standard series 10 (5 wells), the fluorescence intensity at a rabbit IgG concentration of 0 ng/100 μL was considered to be a background fluorescence due to interaction of the autofluorescence of the membrane with BSA diluting the rabbit IgG because this fluorescence intensity was obtained even without the rabbit IgG protein immobilized on the membrane.

Therefore, the net fluorescence intensity of the standard series excluding the background fluorescence is calculated by the following equation:

(Net fluorescence intensity of standard)=(Fluorescence intensity obtained from the standard series)−(Fluorescence intensity obtained from the standard at 0 ng/100 μL).

Example 1 resulted in an average fluorescence intensity obtained from the sample series of 4,061.6 counts and an average fluorescence intensity obtained from the negative control series of 563.6 counts, and therefore, the net fluorescence intensity of the sample series was 3,498 counts.

Example 1 also resulted in a fluorescence intensity obtained from the standard at a rabbit IgG concentration of 0 ng/100 μL of 378 counts, and therefore the net fluorescence intensity of the standard series at the concentrations other than a rabbit IgG concentration of 0 ng/100 μL was as shown in Table 1, which calculates an approximate expression from a fluorescence intensities corresponding to the concentrations of IgG.

TABLE 1
Fluorescence Intensity of Standard Series

IgG concentration [ng/100 μl]

| | 2 | 4 | 8 | 16 |
|---|---|---|---|---|
| Fluorescence intensity | 367.8 | 849.6 | 2316.4 | 4394.6 |
| [counts] | | | | |

A linear approximate expression was obtained from the IgG concentration and the corresponding fluorescence intensity. The linear approximate expression of Example 1 was:

Fluorescence intensity=283.16×(IgG concentration).

The application of the fluorescence intensity of the sample of 3,498 counts to this expression gave an IgG concentration of 12.4 ng/100 μg total proteins.

### Example 2 (see FIG. 4)

1. In an ice bath, HeLa cells (carcinoma cells of uterine cervix) were lysed in a lysis buffer containing 0.1 w/v % NP-40 (produced by Calbiochem), 50 mM Tris-HCl, pH 7.4, 5 mM EDTA, 50 mM sodium fluoride, 1 mM sodium orthovanadate and a protease inhibitor cocktail (produced by Sigma), under the condition of 1×10⁷ cells/5 mL the lysis buffer (pretreatment liquid), by 10 times repeated sucking and discharging with a 5-mL syringe provided with a 23G needle. A cell lysate was thus prepared.

2. Insolubles were removed by centrifugation at 4° C. at 15,000 rpm for 5 minutes. The total amount of proteins contained in the supernatant was measured by the DC protein kit (Bio-Rad) using bovine IgG as reference.

3. A sample liquid was prepared by dilution with TBS (50 mM Tris-HCl, 100 mM NaCl; pH7.4) containing 0.001% NP-40 so that the total amount of proteins became 1 μg/100 μL.

4. A PVDF (polyvinylidene fluoride) membrane was immersed in a transfer buffer (48 mM Tris, 39 mM glycine, 20% methanol, 0.1% SDS and 80% water) for pretreatment.

5. A plate provided with 18 wells (6 lines×3 rows) each having a fixed bottom area (24 mm²) was attached on the pretreated PVDF membrane and fixed so that the bottom of the wells was formed of the PVDF membrane.

6. Each well in 6 lines×1 row of the plate was fed with 100 μL of a sample solution of cultured cells (HeLa) containing 1 μg/100 μL of the total proteins in TBS containing 0.001% of NP-40 (Sample Series 11).

7. Each well in other 6 lines×2 rows of the same plate was fed with a solution of a pure specimen of the target protein of 5 concentrations including 0 ng/100 μL in TBS containing 0.001% of NP-40 and 1 μg/100 μL of BSA, 100 μl each (Standard Series 12). The standard series 12 was of the same specimen on the same plate, and different target proteins were measured using corresponding standard series and different plates.

8. After all the wells of the plate were fed with the liquids, the wells were sucked from the bottom of the wells, that is, from the back face of the membrane at a negative pressure of about 200 mHg for about 15 seconds.

9. Subsequently, all the wells of the plate were fed with a washing liquid (TBS-T: 250 mM Tris, 1.5M aqueous solution of sodium chloride, 1.0% Tween 20), and the wells were sucked from the bottom of the wells at a negative pressure of about 500 mHg for about 30 seconds.

10. All the wells of the plate were fed with a blocking liquid (TBS-T, 4% BSA), 100 μL each, and allowed to stand at room temperature for about 30 minutes. Thereafter, the wells were sucked from the bottom of the wells at a negative pressure of about 500 mHg for about 15 seconds. Then, all the wells of the plate were washed in the same manner as in step 9.

11. All the wells of the plate on which the specimen of the target protein was adsorbed were fed with a solution of a corresponding rabbit antibody (first antibody) which binds specifically to the specimen of the target protein, 100 μL each, and were allowed to stand at room temperature for about 30 minutes.

TABLE 2
Target proteins and Kinds of Rabbit Antibodies
Specifically Binding to Corresponding Specimens
of Target proteins

| | Rabbit antibodies | |
|---|---|---|
| | specifically binding to | |
| | corresponding | |
| Target | specimens of target | |
| proteins | proteins | Solvent of solution |
| Cdk2 | rabbit anti-Cdk2 IgG | TBS (5 mM DTT, 50% glycerol) |
| Cdk4 | rabbit anti-Cdk4 IgG | TBS (5 mM DTT, 50% glycerol) |
| Cyclin E | rabbit anti-CyclinE IgG | TBS (5 mM DTT, 50% glycerol) |
| P16 | rabbit anti-P16 IgG | TBS (5 mM DTT, 50% glycerol) |
| P53 | rabbit anti-P53 IgG | TBS (5 mM DTT, 50% glycerol) |
| P21 | rabbit anti-P21 IgG | TBS (5 mM DTT, 50% glycerol) |
| P27 | rabbit anti-P27 IgG | TBS (5 mM DTT, 50% glycerol) |
| C-myc | rabbit anti-C-myc IgG | TBS (5 mM DTT, 50% glycerol) |

Thereafter, the wells were sucked from the bottom of the wells at a negative pressure of about 500 mHg for about 15 seconds. Then, all the wells of the plate were washed by repeating twice the same process as in step 9.

12. Biotinylated anti-rabbit antibodies (second antibodies) (1/100 solutions of biotinylated anti-rabbit IgGs in TBS-T containing 1% of BSA) were put in the wells of all plates. Thereafter, suction was conducted at a negative pressure of about 500 mHg from the bottom of the wells for about 15 seconds. Then, all the wells of the plates were washed by repeating twice the same process as in step 9.

13. A FITC-labeled streptavidin reagent (1/100 FITC-labeled streptavidin) was added to all the wells of the plates, 100 μL each, and allowed to stand at room temperature for about 30 minutes. Thereafter, suction was conducted at a negative pressure of about 500 mHg from the bottom of the wells for about 15 seconds. Then, all the wells of the plates were washed by repeating three times the same process as in step 9.

14. The PVDF membranes were taken away from the plates, washed with distilled water and then dried at room temperature for about 15 minutes. Thereafter, the PVDF membranes were tested by a fluorometer to measure fluorescence emitted by the substance labeling the proteins adsorbed in a size corresponding to the bottom area of the wells.

15. The quantity of the target proteins in the samples was calculated on the basis of the average fluorescence intensity obtained from the sample series 11 (6 wells) and the fluorescence intensity obtained from the wells at the varied concentrations of the standard series 12 (6 wells×2 rows).

In the standard series, the average fluorescence intensity at a concentration of 0 (2 wells) was considered to be a background fluorescence due to the autofluorescence of the membranes because this fluorescence intensity was obtained even without the target proteins adsorbed on the membranes.

Therefore, the net fluorescence intensity of the samples exclusive of the background fluorescence is calculated by the following equation:

(Net fluorescence intensity of sample)=(Average fluorescence intensity obtained from the sample series)−(Average fluorescence intensity obtained at a concentration of 0 in the standard series).

The fluorescence intensity obtained from the standard series of the varied concentrations of the specimens of the target proteins is shown below, graphs showing calibration lines obtained from the fluorescence intensity of the standard series are shown in the attached figures, and the measured fluorescence intensity of the samples and the calculated concentrations of the target proteins are also shown below.

(i) Measurement of Cdk2

TABLE 3
Concentrations of Target protein and Fluorescence Intensity
Obtained from Standard Series of Specimen

| | Cdk2 (ng/well) | | | | |
|---|---|---|---|---|---|
| | 0.00 | 2.00 | 5.00 | 10.00 | 15.00 |
| Fluorescence | 1285.59 | 1635.78 | 2116.93 | 3062.92 | 3735.63 |
| intensity | | | | | |
| (counts) | | | | | |

(ii) Measurement of Cdk4

TABLE 4
Concentrations of Target protein and Fluorescence
Intensity Obtained from Standard Series of Specimen

Cdk4 (ng/well)

| | 0.00 | 2.00 | 5.00 | 10.00 |
|---|---|---|---|---|
| Fluorescence | 1450.98 | 2314.64 | 3374.60 | 4776.19 |
| intensity | | | | |
| (counts) | | | | |

(iii) Measurement of Cycline E

TABLE 5
Concentrations of Target protein and Fluorescence Intensity
Obtained from Standard Series of Specimen

| | Cycline E (ng/well) | | | | |
|---|---|---|---|---|---|
| | 0.00 | 20.00 | 50.00 | 100.00 | 150.00 |
| Fluorescence | 1920.53 | 5798.08 | 13318.06 | 27332.13 | 34423.52 |
| intensity | | | | | |
| (counts) | | | | | |

(iv) Measurement of P16

TABLE 6
Concentrations of Target protein and Fluorescence Intensity
Obtained from Standard Series of Specimen

| | P16 (ng/well) | | | | | |
|---|---|---|---|---|---|---|
| | 0.00 | 1.00 | 2.50 | 5.00 | 7.50 | 10.00 |
| Fluorescence | 419.68 | 523.54 | 764.21 | 1064.81 | 1309.83 | 1615.15 |
| intensity | | | | | | |
| (counts) | | | | | | |

(v) Measurement of P53

TABLE 7
Concentrations of Target protein and Fluorescence Intensity
Obtained from Standard Series of Specimen

| | P53 (ng/well) | | | | | |
|---|---|---|---|---|---|---|
| | 0.00 | 20.00 | 50.00 | 100.00 | 150.00 | 200.00 |
| Fluo- | 1185.37 | 3232.07 | 6548.63 | 12491.52 | 19104.03 | 23137.77 |
| res- | | | | | | |
| cence | | | | | | |
| inten- | | | | | | |
| sity | | | | | | |
| (counts) | | | | | | |

(vi) Measurement of P21

TABLE 8
Concentrations of Target protein and Fluorescence Intensity
Obtained from Standard Series of Specimen

| | P21 (ng/well) | | | | | |
|---|---|---|---|---|---|---|
| | 0.00 | 20.00 | 50.00 | 100.00 | 150.00 | 200.00 |
| Fluorescence | 599.48 | 882.68 | 1101.06 | 1465.83 | 1966.69 | 2379.38 |
| intensity | | | | | | |
| (counts) | | | | | | |

(vii) Measurement of P27

TABLE 9
Concentrations of Target protein and Fluorescence Intensity
Obtained from Standard Series of Specimen

| | P27 (ng/well) | | | | | |
|---|---|---|---|---|---|---|
| | 0.00 | 2.00 | 5.00 | 10.00 | 15.00 | 20.00 |
| Fluorescence | 503.99 | 823.53 | 1520.01 | 2254.76 | 2722.69 | 3528.19 |
| intensity | | | | | | |
| (counts) | | | | | | |

(viii) Measurement of C-myc

TABLE 10
Concentrations of Target protein and Fluorescence Intensity
Obtained from Standard Series of Specimen

| | C-myc (ng/well) | | | | | |
|---|---|---|---|---|---|---|
| | 0.00 | 2.00 | 5.00 | 10.00 | 15.00 | 20.00 |
| Fluorescence | 431.05 | 917.95 | 1964.85 | 3343.28 | 4687.79 | 5919.69 |
| intensity | | | | | | |
| (counts) | | | | | | |

TABLE 11

| | Fluorescence | | |
|---|---|---|---|
| | intensity of | Quantity in | Concentration in |
| | samples | samples | samples |
| Target proteins | (counts) | (ng/well) | (ng/1 μg) |
| Cdk2 | 1121.8668 | 6.641511 | 6.641510509 |
| Cdk4 | 413.61338 | 0.853098 | 0.853098498 |
| Cycline E | 551.37006 | 1.81718 | 2.022433168 |
| P16 | 461.820014 | 3.762923 | 3.762923089 |
| P53 | 266.630028 | 2.926445 | 2.926444763 |
| P21 | 602.132563 | 63.84379 | 63.84378535 |
| P27 | 476.490014 | 2.49423 | 2.494230426 |
| C-myc | 140.0750042 | 0.355846 | 0.410197873 |

### Effect of the Invention

According to the present invention, a liquid of solubilized cells and tissues is brought in direct contact with a solid phase so that proteins in the liquid are bound to the solid phase, the quantity of a target protein to be assayed is determined using a substance which binds specifically to the target protein. Therefore, the quantity of the target protein can be determined by a reduced number of steps in a short time. Also the quantity of a plurality of target proteins can be determined by a simple method at the same time.

What is claimed is:

1. A method for determining the quantity of a protein comprising the steps of:
(a) preparing a crude protein liquid containing crude proteins by solubilizing tissue or cells in a pretreatment liquid;
(b) contacting the prepared crude protein liquid with a solid phase to allow the crude proteins to bind to the solid phase; and
(c) determining the quantity of a target protein in the crude proteins binding to the solid phase by using a substance which binds specifically to the target protein.

2. The method according to claim 1, wherein the pretreatment liquid contains a surfactant.

3. The method according to claim 2, wherein a concentration of the surfactant in the pretreatment liquid is 1.0 to 0.01 w/v %.

4. The method according to claim 1, wherein the pretreatment liquid contains a protease inhibitor.

5. The method according to claim 1, wherein the solid phase comprises a porous membrane or beads.

6. The method according to claim 1, wherein the substance which binds specifically to the target protein is an antibody or a nucleic acid.

7. The method according to claim 1, wherein the target protein is a water-soluble protein.

8. The method according to claim 1, wherein the target protein is at least one of actin, Cdk1, Cdk2, Cdk4, Cdk6, Cycline B, Cycline D, Cycline E, P16, P21, P27 and C-myc.

9. The method according to claim 1, wherein the step (b) comprises the steps of putting the crude protein liquid in one or more wells of a plate provided with a porous membrane as the solid phase at the bottom of the wells and allowing the crude proteins to bind to the porous membrane by suction from a porous membrane side of the plate.

10. The method according to claim 9, wherein the porous membrane is a hydrophobic porous membrane.

11. The method according to claim 9, wherein the substance which binds specifically to the target protein is an antibody or a nucleic acid.

12. The method according to claim 9, wherein the plate has two or more wells.

13. The method according to claim 12, wherein the step (c) comprises the step of supplying the wells with the substance which binds specifically to the target protein, a first one of the wells is supplied with a substance which binds specifically to a first target protein, and a second one of the wells is supplied with a substance which binds specifically to a second target protein.

14. The method according to claim 13, which is for determining the quantity of two or more proteins.

15. A diagnostic method for diagnosing a disease, the method comprising the steps of:
(a) preparing a crude protein liquid containing crude proteins by solubilizing tissue or cells in a pretreatment liquid;
(b) contacting the prepared crude protein liquid with a solid phase to allow the crude proteins to bind to the solid phase; and
(c) determining the quantity of a target protein in the crude proteins binding to the solid phase by using a substance which binds specifically to the target protein; and
(d) diagnosing a disease according to the determined quantity of the target protein.

16. The diagnostic method according to claim 15, wherein the step (b) comprises the steps of putting the crude protein liquid in one or more wells of a plate provided with a porous membrane as the solid phase at the bottom of the wells and allowing the crude proteins to bind to the porous membrane by suction from a porous membrane side of the plate.

17. The diagnostic method according to claim 15, wherein the disease is stomach cancer, intestinal cancer, breast cancer, lung cancer, esophagus cancer, prostate cancer, liver cancer, kidney cancer, bladder cancer, skin cancer, uterine cancer, brain tumor, osteosarcoma or myeloma.

18. The diagnostic method according to claim 15, wherein the target protein is at least one of actin, Cdk1, Cdk2, Cdk4, Cdk6, Cycline B, Cycline D, Cycline E, P16, P21, P27 and C-myc.

19. A kit for determining the quality of a protein comprising:
a pretreatment liquid for solubilizing tissue or cells;
a solid phase for binding crude proteins in a crude protein liquid prepared by solubilizing the tissue or cells thereto; and
a substance which binds specifically to a target protein in the crude proteins.

20. The kit according to claim 19, wherein the solid phase is a porous membrane or beads.
